(19) United States
(12) Patent Application Publication
(10) Pub. No.: US 2003/0028161 A1
(43) Pub. Date: Feb. 6, 2003

(54) Non-intrusive urine collection apparatus

(76) Inventor: Cecilia Carballo, Miami, FL (US)

Correspondence Address: Chandrakant C. Shroff, Longacre & Associates, Suite 402, 1919 South Eads Street, Arlington, VA, 22202, US

(21) Appl. No.: 09/919,963

(22) Filed: Aug. 2, 2001

(51) Int. Cl. A61F 5/44; A61F 13/15; A61F 13/20

(52) U.S. Cl. 604/349; 604/348; 604/351; 604/353; 604/355; 604/385.09

(57) ABSTRACT

An apparatus is disclosed that allows a caregiver to attach and then, when necessary, dispose of a bag that collects a patient's urine. The bag has an orifice surrounded by an elasticized portion that allows the penis to be inserted and then maintained in position. In the first embodiment of the invention, the bag is held by an adhesive or other type of attachment means to the outside of a diaper-like garment that includes an absorbent inner liner. In the second embodiment, the bag is kept and held inside a standard-type male undergarment (boxers or briefs). In the third embodiment, the bag is held in position outside the typical undergarment. In all embodiments, various types of attachment means or adhesives may be used

### BACKGROUND OF THE INVENTION

1. Field of the Invention

The present invention relates generally to garments that aid in the collection of bodily waste. More specifically, it relates to a garment that can be employed by caregivers to a bedridden person that allows for the collection of urine without the obvious urinal or catheter. Even more specifically, it relates to an undergarment that allows for the collection of urine from a male patient utilizing a bag and an aperture that surrounds and holds the male genitalia to prevent leakage or spillage and that is combined with an undergarment such that there is no obvious visible evidence that the patient is wearing such a garment.

2. Description of the Prior Art

In the case of a bedridden, substantially immobile patient, one of the concerns of the caregivers is the dignity of the person thus affected. Catheterization can be a source of infection and also is an uncomfortable and unnecessary process if the patient is conscious and in control of their bodily functions such as elimination. Since the kidneys are constantly filtering the blood, urination is more frequent than bowel movements. The present invention seeks to provide caregivers with an option other than either catheterization or the use of bedpans, the first, as mentioned above, being uncomfortable and the second being embarrassing for both the patient and the caregiver. Additionally, the present invention is not apparent to any visitors or passers-by so as to minimize the embarrassment and ensuing psychological stress. The invention provides a way in which the patient's penis may be inserted into a urine retaining receptacle and the receptacle may then be emptied discreetly when necessary by a caregiver without any undue embarrassment or complicated steps.

During a search at the U.S. Patent and Trademark Office, a number of relevant patents were uncovered and they will be discussed below.

In U.S. Pat. No. 6,007,524 issued to Jerome Schneider on Dec. 28, 1999 there is disclosed fluid collection and disposal system.

U.S. Pat. No. 5,792,132 issued on Aug. 11, 1998 to Lucia Garcia discloses an incontinence diaper system.

U.S. Pat. No. 5,843,065 issued to James Wyant on Dec. 1, 1998 describes an incontinence diaper with an opening.

U.S. Pat. No. 5,716,350 issued on Feb. 10, 1998 to Richard T. Ryan discloses a medical protection device for males.

U.S. Pat. No. 6,059,762 issued to Mildred E. Boyer et al. on May 9, 2000 discloses an incontinence device for males.

U.S. Pat. No. 5,569,229 issued on Oct. 29, 1996 to Candies Rogers discloses a diaper with a view flap.

U.S. Pat. No. 2,538,758 issued to George Bricmont on Jan. 23, 1951 discloses a diaper device.

U.S. Pat. No. 5,984,910 issued on Nov. 16, 1999 to Joseph Berke teaches an incontinence device and method.

Lastly, U.S. Pat. No. 5,618,279 issued to Edward Pudlo on Apr. 8, 1997 discloses a medical protection device for males.

Thus, while the foregoing overview of prior art indicates it to be well known to provide various incontinence protection devices, diapers, and the like, none of the inventions discussed above, either alone or in combination, describe the instant invention as claimed.

### SUMMARY OF THE INVENTION

To achieve the foregoing and other advantages, the present invention, briefly described, provides an apparatus that allows a caregiver to attach and then, when necessary, dispose of a bag that collects a patient's urine. The bag has an orifice surrounded by an elasticized portion that allows the penis to be inserted and then maintained in position. In the first embodiment of the invention, the bag is held by an adhesive or other type of attachment means to the outside of a diaper-like garment that includes an absorbent inner liner. In the second embodiment, the bag is kept and held inside a standard-type male undergarment (boxers or briefs). In the third embodiment, the bag is held in position outside the typical undergarment. In all embodiments, various types of attachment means or adhesives may be used.

Thus it is a principal object of the invention to provide a non-intrusive urine collection apparatus that rids a bedridden patient of the embarrassment, danger and discomfort of catheterization,

It is a further object of the invention to provide a non-intrusive urine collection apparatus that is not visible to visitors or passers-by.

Still yet a further object of the invention is to provide a non-intrusive urine collection apparatus that includes a urine collection bag having an elasticized orifice that holds the patient's penis in place.

Yet another object of the invention is to provide a non-intrusive urine collection apparatus that includes various attachment means for holding the collection bag in place through hook and loop type fasteners, snaps, drawstrings, belts, or the like.

Still yet another object of the invention is to provide a non-intrusive urine collection apparatus that may be provided in various sizes for both children, infants, or adults.

And still yet another object of the invention is to provide a non-intrusive urine collection apparatus that is inexpensive to manufacture and thus may be widely provided at low cost to the public.

These together with still other objects of the invention, along with the various features of novelty which characterize the invention, are pointed out with particularity in the claims annexed to and forming a part of this disclosure. For a better understanding of the invention, its operating advantages and the specific objects attained by its uses, reference should be made to the accompanying drawings and descriptive matter in which there are illustrated preferred embodiments of the invention.

### BRIEF DESCRIPTION OF THE DRAWINGS

The invention will be better understood and the above objects as well as objects other than those set forth above will become more apparent after a study of the following detailed description thereof. Such description makes reference to the annexed drawings wherein:

FIG. 1 is a view of the first embodiment of the invention showing the diaper-like portion of the apparatus.

FIG. 2 is a partial side view of the first embodiment of the invention showing the collection bag being held in place outside of the diaper-like undergarment.

FIG. 3 is a perspective view of the generic bag used in the instant invention showing the elasticized aperture and (in one contemplated practice) the hook and loop type fasteners that keep the bag in the desired location.

FIG. 4 is a view of the second embodiment of the instant invention wherein the collection bag is located inside the undergarment.

FIG. 5 is a view of the third embodiment of the invention wherein the collection bag is held in place outside of the undergarment.

### DESCRIPTION OF THE PREFERRED EMBODIMENTS

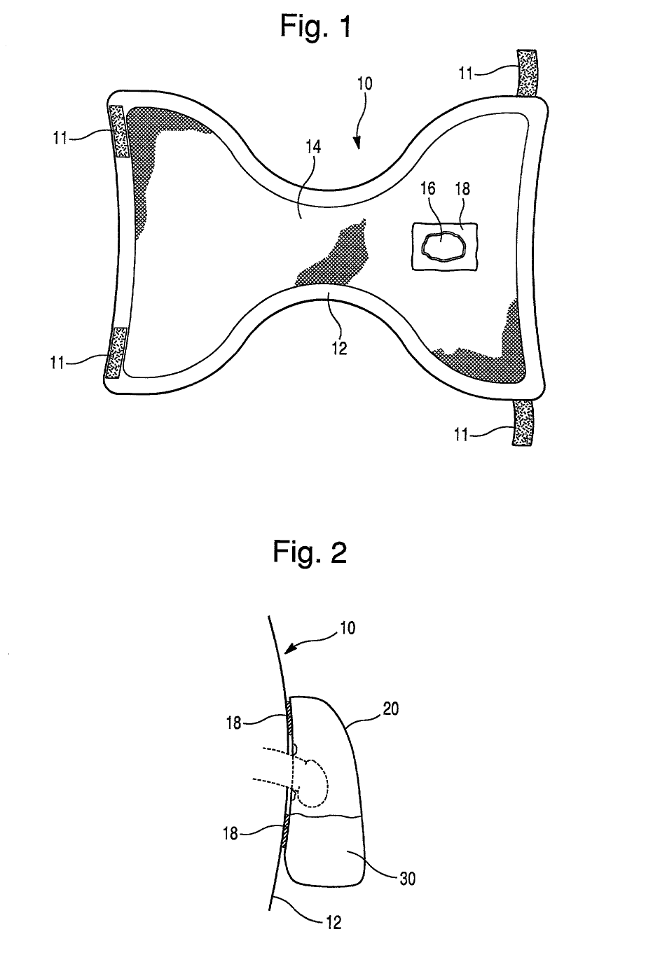

The three embodiments will be discussed one at a time. First, turning to FIG. 1, the diaper portion of the first embodiment of the instant invention is generally designated at 10. It should be understood that the inner portion of the undergarment is what is seen in the figure. The garment could be made of any number of various materials, but it is contemplated that the outer layer 12 would preferably be made from either a liquid resistant polymer or a highly absorbent fiber. In either case, the inner layer 14 would be made of an even more absorbent material such that excreta would be carried away from the patient's body in the interim before a caregiver replaced the garment. Note that garment 10 is held in place on the patient's body (in the embodiment described in the Figure) by straps and cooperating patches indicated at 11. Other ways of fixing garment 10 onto the patient would, of course, be obvious to the skilled practitioner. Located in the area of the garment 10 designed to be proximate the pubis of the patient is an aperture 16. Aperture 16 is surrounded (in the embodiment described herein) by an adhesive area indicated at 18. It should be noted that this adhesive area would be located on the outer side of the garment 10 on the outer layer 12. Turning to FIG. 2, it can be seen that the adhesive area 18 holds the bag 20 in place against the outer layer 12 of the diaper-like garment 10.

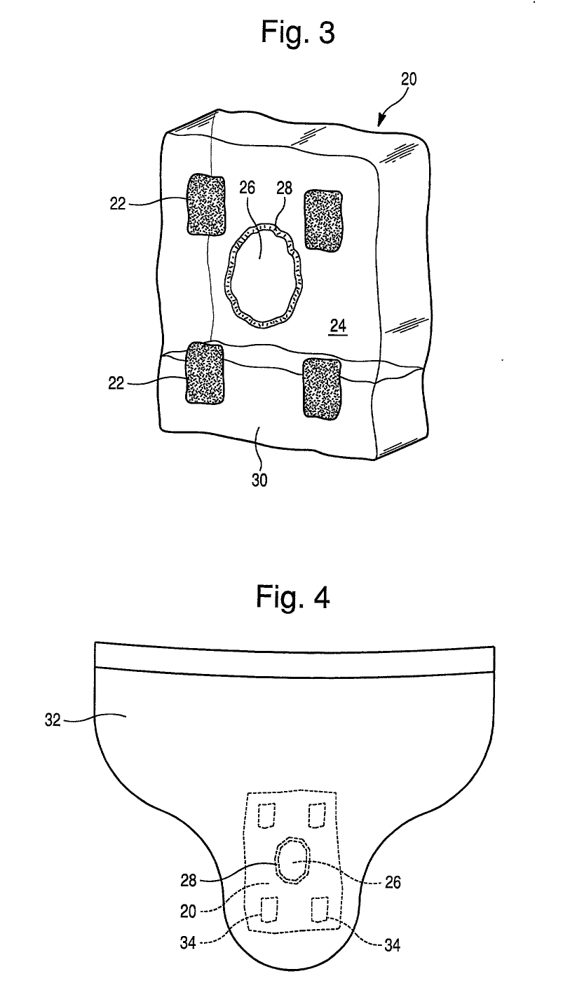

Turning to FIG. 3, the discussion will now focus on the collection bag 20. In the contemplated embodiment seen in FIG. 3, the bag 20 includes hook and loop type fasteners 22 located on the face 24 of the bag 20 that will be most proximate the patient's pubis. This allows the bag to be located and held in place on the outer layer 12 of the garment 10. It should be emphasized that many other types of fasteners could be used in place of the hook and loop type seen here. Snaps, drawstrings, belts, or chemical adhesives: all of these could easily be used without departing from the spirit of the invention. Located on the same face 24 of the bag 20 is an aperture 26. Surrounding aperture 26 is an elasticized portion 28 that allows the patient's penis to be held firmly in place so as not to be dislodged during movement. Even though the patient that the present invention is designed for would be substantially immobile, the normal caregiving procedure does involve movement during bathing, changing the sheets, or the like, so that this feature of the instant invention is important and is included in all the embodiments. Bag 20 could be made out of any number of materials, so long as the materials were leakproof and strong enough to resist tearing during movement. Urine, indicated at 30, is collected in the bottom of the bag 20. When a sufficient amount has been collected bag 20 is detached by the caregiver and replaced with a new one. Please note that in the first embodiment, seen in FIGS. 1 and 2, that garment 10 and bag 20 could be monolithic, that is a single piece, and that aperture 16, seen in FIG. 1, could be surrounded by the elasticized portion. Thus, both the diaper-like garment and the collection bag would be replaced at the same time.

The second embodiment of the instant invention will now be discussed. This is seen in FIG. 4. In this embodiment bag 20 is maintained in place inside the undergarment, seen here as what is commonly referred to a “jockey” type shorts indicated at 32. The bag 20 with aperture 26 is held in place by attachment means indicated at 34. Once again, these could be any number of types of attachments, including hook and loop type fasteners, chemical adhesives, belts, drawstrings or the like. The appropriate cooperating feature (where necessary) of the attachment means would be located on the interior of the shorts 32. In use, the shorts 32 would be lowered slightly, the patient's penis would be inserted into the aperture 26, held in place by the elasticized portion 28 and then the bag 20 would be attached to the interior of the shorts 32. It should be mentioned at this juncture that the bag 20 does not necessarily have to be fastened to the shorts 32 if it is not desired or necessary. The shorts 32 themselves could hold the bag 20 in an appropriate position if the patient was substantially immobile.

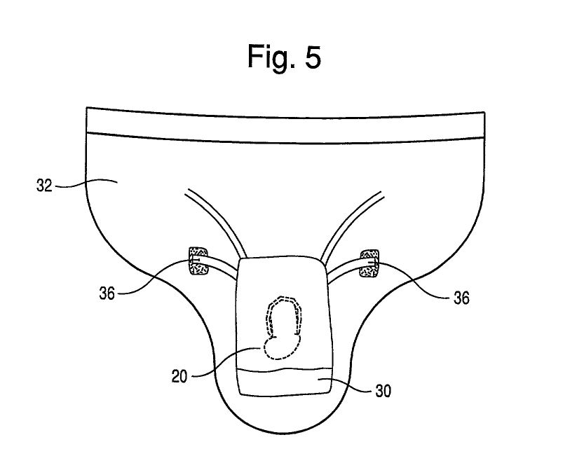

In the third embodiment of the invention seen in FIG. 5, the bag 20 is located on the exterior of the shorts 32. In the type of the shorts seen herein, a “fly” portion or slit is commonly provided for the ease in urination. The patient's penis may be passed through this slit and then through the aperture 26 and held by the elasticized portion 28. Bag 20 may then be attached to the outer portion of the shorts 32 by attachment means 36. Attachment means 36 are seen in the Figure to be hook and loop type fasteners but, again, it should be emphasized that many other types of attachment or adhesive mechanisms could be used to bring the instant invention into practice.

It should be emphasized that the instant invention is not in any way limited to the embodiments as they are described above but encompasses all embodiments as described in the scope of then following claims.

What is claimed as being new and desired to be protected by Letters Patent of the United States is as follows:

1. A non-intrusive urine collection apparatus for a substantially bed-ridden male patient comprising:
a collection bag, said collection bag including an aperture for the reception of a patient's penis, said aperture further including a surrounding portion to aid in the holding of the patient's penis in the confines of said aperture; and
garment attachment means for fixing said collection bag in a desired position in relation to an undergarment worn by the patient; whereby
the patient's penis is placed in said aperture so that urine may be collected in the interior of said collection bag and said collection bag is fixed in a desired location through said garment attachment means such that said collection bag and said undergarment are held in a fixed relationship to one another and said collection bag may be removed and replaced without substantial inconvenience to the patient.

2. The apparatus according to claim 1, wherein said undergarment further includes an inner layer and an outer layer and where said inner layer comprises a highly absorbent material for carrying fecal away from the body of the patient.

3. The apparatus according to claim 2 wherein said outer layer comprises a substantially leakproof liquid barrier and further wherein said undergarment further includes a garment aperture located in the area proximate the patient's pubis.

4. The apparatus according to claim 3, wherein said collection bag is attached, through said garment attachment means, to said outer layer of said undergarment.

5. The apparatus according to claim 4, wherein said garment attachment means comprises hook and loop type fasteners.

6. The apparatus according to claim 1, wherein said undergarment comprises jockey type shorts or boxer type shorts and where said collection bag is fixed through said garment attachment means interiorly of said jockey shorts or boxer shorts.

7. The apparatus according to claim 6, wherein said garment attachment means comprises hook and loop type fasteners.

8. The apparatus according to claim 1, wherein said undergarment comprises jockey type shorts and where said collection bag is fixed through said garment attachment means exteriorly of said jockey shorts.

9. The apparatus according to claim 8, wherein said garment attachment means comprises hook and loop type fasteners.
